(19) United States
(12) Patent Application Publication
(10) Pub. No.: US 2021/0375481 A1
(43) Pub. Date: Dec. 2, 2021

(54) TRACING CONTACTS AMONG A GROUP OF INDIVIDUALS

(71) Applicant: INFORM Software Corporation, Atlanta, GA (US)

(72) Inventor: Adrian Weiler, Aachen (DE)

(21) Appl. No.: 16/886,234

(22) Filed: May 28, 2020

(51) Int. Cl. G16H 50/80; G16H 10/65; H04W 12/02; H04W 4/02

(52) U.S. Cl. CPC G16H 50/80; G16H 10/65; G06Q 10/105; H04W 4/023; H04W 12/02

(57) ABSTRACT

Tracing device for tracing contacts among a group of individuals in order to contain the spreading of an infectious disease, wherein the tracing device s configured to:

a) transmit a respective individual identifier,

b) receive the individual identifiers from other tracing devices in reach, and

c) store individual identifiers received in b) in a log,

and wherein the log of the tracing device can be read by a reading device.

### BACKGROUND

The invention is directed to a tracing device, a use, an arrangement and a method for tracing contacts among a group of individuals in order to contain the spreading of an infectious disease.

Infectious diseases such as Covid-19 are spread by droplet infection. Hence, in order to contain the spreading of such a disease it is important to know who an infected person met while being contagious. From the prior art, systems are known that are supposed to trace contacts of people. However, these systems usually require a central control station. This is complicated, expensive and undesirable in terms of data privacy.

### SUMMARY

The object of the invention is to provide a tracing device, a use, an arrangement and a method for tracing contacts among a group of individuals in order to contain the spreading of an infectious disease that are simple, inexpensive and that allow a particularly high standard of data privacy.

The object is solved with a tracing device, a use, an arrangement and a method according to the independent claims. Advantageous refinements are presented in the dependent claims. The features described in the claims and in the description can be combined with each other in any technologically reasonable manner.

According to the invention a tracing device for tracing contacts among a group of individuals in order to contain the spreading of an infectious disease is presented. The tracing device is configured to
a) transmit a respective individual identifier,
b) receive the individual identifiers from other tracing devices in reach, and
c) store individual identifiers received in b) in a log,
   wherein the log of the tracing device can be read by a reading device.

With the described tracing device spreading of an infectious disease such as Covid-19 can be contained. Therefore, contacts among individuals of a group of individuals can be traced using the tracing device. A “contact” of two individuals is an incident in which the two individuals came into proximity of each other. It is not necessary that the two individuals came into physical contact, i.e. a touch is not required. The tracing device is supposed to be used in combination with other tracing devices. Preferably, the other tracing devices are configured identically to the described tracing device. Preferably, individuals of a closed group of individuals such as employees of a company are provided with a respective tracing device.

The tracing device is capable of providing a log showing which of the other tracing devices has been in the proximity of the described tracing device. Preferably, the log is a log file. Alternatively, the log can be a configured as a blockchain. The log can be considered a database of past contacts of the individual assigned the respective tracing device. If each individual of a group of individuals has a respective tracing device, this allows determination of contacts among this group of individuals. This information can help contain the spreading of an infectious disease. Therefore, no central control station is required. It is, thus, preferred that the tracing device is configured to be used with other tracing devices in a decentralized manner. In particular, the tracing device is preferably configured to be used independently of a central control station such as a server. The tracing device is preferably configured not to be in frequent contact with a server. In view of data privacy, it is preferable that the tracing device is only capable of what is described herein. In particular, it is preferably that the tracing device is not a mobile phone.

The tracing device preferably comprises a DSP (digital signal processor) that controls the described functionality. Further, the tracing device preferably comprises a memory, in which the log can be stored. The tracing device is preferably powered by a battery. Further, the tracing device preferably comprise an antenna for transmitting the individual identifier and receiving the individual identifiers from the other tracing devices in reach.

The tracing device is capable of transmitting a respective individual identifier. Preferably, the individual identifier is an individual identification number. Alternatively, the individual identifier can be an individual identification code that can comprise numbers, letter and special characters, e.g. such as in a MAC address of the device. Each tracing device has its own, unique individual identifier. The individual identifier can be considered a registration code of the tracing device. Hence, the tracing device can be assigned unambiguously to a user. A mapping of the individual identifiers and the respective users is preferably provided at a secure location. This might be, for example, the human resource department of a company. Except for this mapping, all information gathered with the tracing device is anonymous.

In order for other tracing devices to be capable of recognizing the described tracing device, the described tracing device transmits its individual identifier so that the other tracing devices can receive it. The transmission may be continuous or discontinuous, for example periodical. In addition or alternatively it is possible that the individual identifier is transmitted upon request of another tracing device. The individual identifier is transmitted wirelessly.

The described tracing device is capable of receiving the individual identifiers from other tracing devices in reach. That is, the described tracing device can receive the individual identifiers from those tracing devices that are within the reach of the transmission method. The reach can be, for example, 10 m [meter] through air. In case there are obstructions such as walls, the reach can be reduced. The power of a transmitted signal usually decreases with the distance from the transmitting tracing device. Hence, the fact that a transmitting tracing device is in reach means that the at the location of the receiving tracing device the power of a signal transmitted from the transmitting device is larger than a threshold value. The threshold value is defined such that the signal can be received and processed properly.

Once received, the individual identifiers are stored in a respective log. The log is stored within the described tracing device, in particular in a memory of the described tracing device. That is, in normal operation the log is kept within the tracing device. Preferably, in normal operation no log entry leaves the tracing device. This is advantageous in terms of data privacy. Normal operation is the time during which contacts are traced. Normal operation is distinguished, in particular, from reading the log using a reading device.

The log is preferably read by the reading device in case the individual assigned the described tracing device is diagnosed with the infectious disease. In particular, it is preferred that the log is read only in case the respective individual is diagnosed with the infectious disease. It is sufficient to read the log of the tracing device of the infected individual. The log of the tracing devices of other individuals, including those who had contact with the infected individual, do not have to be read. The reading device can read the log such that the individual identifiers of those individuals can be determined that were in proximity with the diagnosed individual. This is preferably limited to a certain period of time before the diagnosis. This is preferably adapted to the incubation time of the infectious disease. In the case of Covid-19, for example, a period of 14 days may be used.

The reading device preferably communicates with the tracing devices wirelessly, for example via Bluetooth. The reading device can be, for example, a mobile phone equipped with a suitable app. Once the log is read by the reading device, the reading device can display the log entries or forward them to other electronic devices. From the displayed or forwarded individual identifiers those individuals can be determined that were in contact with the diagnosed individual. These individuals can be warned and/or ordered to stay in quarantine. This can help contain the spreading of the infectious disease.

In a preferred embodiment the tracing device is configured to perform a) and/or b) periodically.

It is sufficient to gather information about contacts among the individuals in time steps of, for example, 10 seconds. Hence, it is not necessary to transmit and receive the individual identifiers continuously. Preferably, the individual identifier is transmitted periodically at a periodicity of between 1 to 60 seconds, in particular between 5 to 20 seconds. In particular in case of a periodic transmission it is preferred that the tracing device is configured to receive individual identifiers continuously. That is, every transmitted individual identifier can be received as long as the transmitting and receiving tracing devices are within reach.

In a further preferred embodiment, the tracing device is configured to further store at least one of the following in c):
a respective time of receipt of the individual identifier,
a respective distance between the receiving tracing device and the transmitting tracing device at the time of receipt of the individual identifier.

Knowing the time of receipt is desirable because an infected individual is contagious only over a certain period of time. Hence, only contacts from this period of time matter. In the present embodiment, therefore, the time of receipt it stored along the individual identifier. That is, each log entry comprises a time stamp.

In containing a disease, it is also helpful to known how long a certain contact occurred. If the individual identifier is transmitted periodically, for example, every 10 seconds, a short contact of 5 seconds will provide at most one log entry. A contact of 10 minutes, in contrast, will most likely provide 60 log entries. If the connection fails temporarily, at least most of these log entries are likely to be made. Hence, as long as the connection does not fail during almost the entire contact, at least sufficient information about the contact will be gathered.

In particular those contacts are of interest that lasted longer than a certain period of time. Shorter contacts may be considered harmless. Hence, it is preferable to be able to identify those contacts that lasted longer than a predetermined period of time. This can be achieved using the log. For example, only those contacts may be processed further that resulted in a certain minimum number of log entries. The minimum number of log entries can be determined from the desired minimum period of time in relation to the periodicity of the transmission of the individual identifier. Alternatively or additionally, the periodicity of the transmission of the individual identifier can be chosen such that certain short-term contacts are unlikely to be recognized.

In a further preferred embodiment the tracing device is configured to store an individual identifier in c) only in case the transmitting tracing device was within a predetermined range from the receiving tracing device at the time of receipt of the respective individual identifier, wherein the predetermined range is smaller than the reach of the transmission in a).

In the spreading of a disease the distance between individuals is important. With respect to Covid-19, for example, a distance of more than 2 m [meter] between individuals is considered save. Hence, it is preferred that the tracing device is configured to receive signals only from those other tracing devices that are within the predetermined range. The predetermined range is preferably 5 m, in particular 2 m [meter]. The predetermined range is smaller than the reach of the transmission. The tracing device can be configured to receive signals only from those other tracing devices that are within the predetermined range by setting a threshold value for the received signal power accordingly. In that case, the threshold value is not only defined such that the signal can be received and processed properly.

In a further preferred embodiment the tracing device is configured to store an individual identifier in c) only in case the transmitting tracing device stayed within a predetermined range from the receiving tracing device over a first predetermined period of time around the time of receipt of the respective individual identifier.

A disease is usually not transferred between individuals that came into contact for less than 15 minutes. Hence, it is reasonable to limit the log entries to contacts in which the individuals were within the predetermined range for at least a certain period of time. This period of time is labelled first period of time in order to distinguish it from other periods of time described herein. The first period of time is preferably 1 minute, in particular 10 minutes or even 15 minutes. The predetermined range is preferably smaller than the reach of the transmission in a). In particular, the predetermined range is preferably 5 m, in particular 2 m [meter].

In a further preferred embodiment the tracing device is configured to delete log entries after a predetermined second period of time.

An infected individual is contagious only over a certain period of time. Hence, contacts from before this time are not relevant for containing the spreading of the infectious disease. In view of data privacy the respective log entries are therefore deleted. Preferably, the tracing device is configured such that the log entries are deleted automatically after a certain period of time. This is labelled second period of time. The second period of time preferably begins when the respective log entry is stored in the log or with the respective time of receipt. Both may be the same since the log entry is preferably stored in the log at the time of receipt. In particular, the second period of time may begin with the respective time stamp of a log entry. For each log entry the second period of time may begin at a different point of time. The second period of time is preferably 20 days, in particular 14 days.

In a further preferred embodiment the tracing device is configured to be activated upon detection of a motion of the respective tracing device.

The tracing device preferably comprises a motion sensor that can detect if the tracing device is in motion. If the tracing device is carried by an individual, the tracing device is usually in motion at least to such an extent that the motion can be detected by a motion sensor. Thus, the tracing device can be activated upon detection of a motion. Preferably, the tracing device is deactivated once a certain period of time has lapsed since the last detected motion. Hence, the tracing device is only active if carried by an individual. It is not necessary for the user to manually switch the tracing device on or off. Also, the automatic deactivation is desirable in view of data privacy since no information is gathered if the tracing device is not carried by an individual. The tracing device, therefore, cannot be misused by hiding the tracing device in order to record who enters a certain the room. Also, if the tracing devices are used by employees it is possible for the employees to take the tracing device home, where it can be deactivated by placing it on a table of into a drawer.

In a further preferred embodiment the tracing device is incapable of determining its own position.

The fact that the tracing device is incapable of determining its own position means that the tracing device has no GPS sensor or similar element. Switching off such an element would not make the tracing device incapable of determining its own position. Instead, in this embodiment the tracing device is permanently incapable of determining its own position. In this embodiment it is not possible to use the tracing device to record a comprehensive motion profile of the individual carrying the tracing device. Such information is not necessary in order to contain the spreading of an infectious disease. In view of data privacy, hence, this information is not gathered. Using the tracing devices, there is no surveillance of the users. The tracing device of an individual is read only in case this individual is infected. In particular in this embodiment the tracing device is distinguished from a mobile phone.

In a further preferred embodiment the tracing device is incapable of communicating other than transmitting the individual identifier according to a) and, upon request by the reading device, providing the log to a reading device.

In view of data privacy it is preferable for the tracing device to store the log securely. Only upon request by the reading device the log can be extracted from the tracing device. Other means of communication, except the transmission of the individual identifier, are unnecessary for containing the spreading of an infectious disease. Hence, in the present embodiment no such communication is allowed or even possible. In particular in this embodiment the tracing device is distinguished from a mobile phone.

In a further preferred embodiment the tracing device is configured such that the log can be read by the reading device only if the reading device is within less than 1 m [meter] of the tracing device.

In this embodiment reading the log is further secured. In particular, the log is not transferred from the tracing device to the reading device via a cellular network, a WLAN network or the internet. Instead, only a short-distance wireless connection is used. For example, a Bluetooth or near field connection can be used.

In a further preferred embodiment the tracing device is configured to transmit the respective individual identifier in a) via Bluetooth.

In a further preferred embodiment the tracing device is smaller than 8 cm [centimeter], in particular smaller than 5 cm in any direction and/or wherein the tracing device has a mass of less than 100 g [gram], in particular less than 50 g.

The combination is preferred in which the tracing device has the described dimension and weight. The tracing device does not have to be capable of performing operations other than those described herein. Hence, it is not necessary that the tracing device is larger or heavier than according to this embodiment. This increases user comfort. In particular in this embodiment the tracing device is distinguished from a mobile phone.

In a further preferred embodiment the tracing device comprising a battery incapable of being recharged.

There is no need to recharge the tracing device or to provide respective charging means. Instead, the tracing device is preferably configured so as to consume only a minimum of energy. Thereby, the tracing device can be used for up to twelve months. During this time, no maintenance of the tracing device is required. Once the battery is empty, the tracing device can be replaced by a new tracing device. The old tracing device can be recycled. In particular in this embodiment the tracing device is distinguished from a mobile phone.

As a further aspect of the invention a use of the described tracing device is presented for tracing contacts among a group of individuals in order to contain the spreading of an infectious disease.

The advantages and features of the described tracing device are transferrable to the described use, and vice versa.

As a further aspect of the invention an arrangement for tracing contacts among a group of individuals in order to contain the spreading of an infectious disease is presented. The arrangement comprises a respective tracing device for each individual, wherein the tracing devices are configured to
a) transmit a respective individual identifier,
b) receive the individual identifiers from the other tracing devices in reach, and
c) store individual identifiers received in b) in a respective log,
   wherein, upon diagnosing one of the individuals with the infectious disease, the log of the tracing device of this individual can be read by a reading device such that other individuals can be identified that had contact with the diagnosed individual.

The advantages and features of the described tracing device and its use are transferrable to the described arrangement, and vice versa. The tracing devices of the arrangement are preferably configured as described. The reading device is preferably not part of the arrangement. However, in an alternatively preferred embodiment the reading device is part of the arrangement.

As a further aspect of the invention a method for tracing contacts among a group of individuals in order to contain the spreading of an infectious disease is presented. Each individual has a respective tracing device that
) transmits a respective individual identifier,
b) receives the individual identifiers from the other tracing devices in reach, and
c) stores individual identifiers received in b) in a respective log,
   wherein, upon diagnosing one of the individuals with the infectious disease, the log of the tracing device of this individual is read by a reading device such that other individuals can be identified that had contact with the diagnosed individual.

The advantages and features of the described tracing device, its use and the described arrangement are transferrable to the described method, and vice versa. The method is preferably performed with tracing devices configured as described. The described tracing device and the described arrangement are preferably configured to be used in the described method.

The tracing devices are preferably handed down to a closed group of people. In a preferred embodiment of the method the individuals are employees of the same employer.

Using the described tracing devices is particularly advantageous in a company because there is no need to use the private mobile phones of the employees, which might violate data privacy regulations. In particular, it might be forbidden for the employer to require employees to install a tracing app on their private mobile phones. Also, it is not necessary to equip every employee with a mobile phone provided by the employer, which would be very expensive.

In a further preferred embodiment of the method the individuals carry the respective tracing device along only during working hours.

In view of data privacy it is desirable for the tracing devices to be used only during working hours and preferably only at the workplace. Tracing is preferably stopped once the employee leaves the workplace.

In a further preferred embodiment of the method only upon diagnosing one of the individuals with the infectious disease the log of the tracing device of this individual is read by the reading device.

If an individual is not diagnosed with the infectious disease, the log of the tracing device of this individual is not read. Only the logs of the tracing devices of diagnosed individuals are read. This further improves data privacy.

### BRIEF DESCRPTION OF THE DRAWINGS

In the following the invention will be described with respect to the figure. The figure shows a preferred embodiment, to which the invention is not limited. The figure and the dimensions shown therein are only schematic. The figure shows:

FIG. 1: an arrangement for tracing contacts among a group of individuals in order to contain the spreading of an infectious disease.

### DETAILED DESCRIPTION

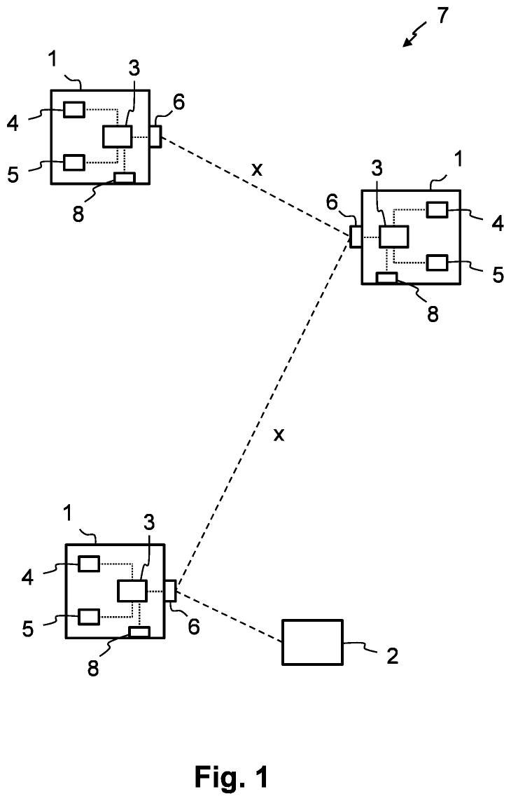

FIG. 1 shows an arrangement 7 for tracing contacts among a group of individuals in order to contain the spreading of an infectious disease. The arrangement 7 comprises a respective tracing device 1 for each individual. Since each tracing device 1 is assigned a respective individual, only the tracing devices 1 are shown and the individuals are not shown. The individuals can be employees of the same employer. The individuals may carry the respective tracing device 1 along during working hours only. The tracing devices 1 are configured to
a) transmit a respective individual identifier via Bluetooth,
b) receive the individual identifiers from the other tracing devices 1 in reach, and
c) store individual identifiers received in b) in a respective log.

The tracing devices 1 comprise a respective DSP 3 (digital signal processor) that controls the described functionality. Further, the tracing devices 1 comprise a respective memory 4, in which the respective log is stored. The tracing devices 1 are powered by a respective battery 5. The battery 5 is incapable of being recharged. Further, the tracing devices 1 comprise a respective antenna 6 for transmitting the individual identifier and receiving the individual identifiers from the other tracing devices 1 in reach. Also, the tracing devices 1 comprise a respective motion sensor 8 for detecting motion of the respective tracing device 1. The memory 4, the battery 5, the antenna 6 and the motion sensor 8 are each connected to the DSP 3 as indicated by dotted lines.

Upon diagnosing one of the individuals with the infectious disease, the log of the tracing device 1 of this individual can be read by a reading device 2 such that other individuals can be identified that had contact with the diagnosed individual. In FIG. 1 a situation is shown in which the reading device 2 is connected to one of the tracing devices 1. In normal operation, however, the reading device 2 is not connected to any tracing device 1.

The tracing devices 1 are configured to perform a) and b) periodically. The tracing devices 1 are also configured to further store in c):
a respective time of receipt of the individual identifier,
a respective distance x between the receiving tracing device 1 and the transmitting tracing device 1 at the time of receipt of the individual identifier. In FIG. 1 the distance x is indicated by dashed lines that also represent signals transferred between the tracing devices 1.

The tracing devices 1 are configured to store received individual identification identifiers in c) only in case the transmitting tracing device 1 stayed within a predetermined range from the receiving tracing device 1 over a first predetermined period of time around the time of receipt of the respective individual identifier. The tracing devices 1 are further configured to delete log entries after a predetermined second period of time. The tracing devices 1 are further configured to be activated upon detection of a motion of the respective tracing device 1. Therefore, the tracing devices 1 comprise the motion sensor 8. The tracing devices 1 are incapable of determining their own positions and of communicating other than transmitting the individual identifier according to a) and, upon request by the reading device 2, providing the log to the reading device 2. Further, the tracing devices 1 are configured such that the log can be read by the reading device 2 only if the reading device 2 is within less than 1 m [meter] of the tracing device 1. Only upon diagnosing one of the individuals with the infectious disease the log of the tracing device 1 of this individual is read by the reading device 2. The tracing devices are smaller than 8 cm [centimeter] in any direction and have a respective mass of less than 100 g [gram].

### LIST OF REFERENCE NUMERALS

1 tracing device
2 reading device
3 DSP
4 memory
5 battery
6 antenna
7 arrangement
8 motion sensor
x distance

1. A tracing device for tracing contacts among a group of individuals in order to contain the spreading of an infectious disease, wherein the tracing device is configured to:
a) transmit a respective individual identifier,
b) receive the individual identifiers from other tracing devices in reach, and
c) store individual identifiers received in b) in a log, and wherein the log of the tracing device can be read by a reading device.

2. The tracing device according to claim 1, wherein the tracing device is configured to perform a) and/or b) periodically.

3. The tracing device according to claim 1, wherein the tracing device is configured to further store at least one of the following in c):
a respective time of receipt of the individual identifier,
a respective distance between the receiving tracing device and the transmitting tracing device at the time of receipt of the individual identifier.

4. The tracing device according to claim 1, wherein the tracing device is configured to store an individual identifier in c) only in case the transmitting tracing device was within a pre-determined range from the receiving tracing device at the time of receipt of the respective individual identifier, wherein the predetermined range is smaller than the reach of the transmission in a).

5. The tracing device according to claim 1, wherein the tracing device is configured to store an individual identifier in c) only in case the transmitting tracing device stayed within a predetermined range from the receiving tracing device over a first predetermined period of time around the time of receipt of the respective individual identifier.

6. The tracing device according to claim 1, wherein the tracing device is configured to delete log entries after a predetermined second period of time.

7. The tracing device according to claim 1, wherein the tracing device is configured to be activated upon detection of a motion of the respective tracing device.

8. The tracing device according to claim 1, wherein the tracing device is incapable of determining its own position.

9. The tracing device according to claim 1, wherein the tracing device is incapable of communicating other than transmitting the individual identifier according to a) and, upon request by the reading device, providing the log to the reading device.

10. The tracing device according to claim 1, wherein the tracing device is configured such that the log can be read by the reading device only if the reading device is within less than 1 m [meter] of the tracing device.

11. The tracing device according to claim 1, wherein the tracing device (1) is configured to transmit the respective individual identifier in a) via Bluetooth.

12. The tracing device according to claim 1, wherein the tracing device is smaller than 8 cm [centimeter] in any direction and/or wherein the tracing device has a mass of less than 100 g [gram].

13. The tracing device according to claim 1, comprising a battery incapable of being re-charged.

14. A system for tracing contacts among a group of individuals in order to contain the spreading of an infectious disease, the system comprising:
a respective tracing device for each individual, wherein the tracing devices are configured to:
a) transmit a respective individual identifier,
b) receive the individual identifiers from the other tracing devices in reach, and
c) store individual identifiers received in b) in a respective log,
and wherein, upon diagnosing one of the individuals with the infectious disease, the log of the tracing device of this individual can be read by a reading device such that other individuals can be identified that had contact with the diagnosed individual.

15. The system of claim 14, wherein the tracing devices are configured to perform a) and/or b) periodically.

16. The system of claim 14, wherein the tracing devices wherein the tracing device is configured to further store at least one of the following in c):
a respective time of receipt of the individual identifier,
a respective distance between the receiving tracing device and the transmitting tracing device at the time of receipt of the individual identifier.

17. A method for tracing contacts among a group of individuals in order to contain the spreading of an infectious disease, wherein each individual has a respective tracing device that is configured to:
a) transmit a respective individual identifier,
b) receive the individual identifiers from the other tracing devices in reach, and
c) store individual identifiers received in b) in a respective log,
and wherein, upon diagnosing one of the individuals with the infectious disease, the log of the tracing device of this individual is read by a reading device such that other individuals can be identified that had contact with the diagnosed individual.

18. The method according to claim 17, wherein the individuals are employees of the same employer.

19. The method according to claim 18, wherein the individuals carry the respective tracing device along during working hours only.

20. The method according to claim 17, wherein only upon diagnosing one of the individuals with the infectious disease the log of the tracing device of this individual is read by the reading device.
